(19) United States
(12) Patent Application Publication
(10) Pub. No.: US 2015/0150995 A1
(43) Pub. Date: Jun. 4, 2015

(54) CONJUGATED ANTI-MICROBIAL COMPOUNDS AND CONJUGATED ANTI-CANCER COMPOUNDS AND USES THEREOF

(71) Applicant: PONO CORPORATION, Honolulu, HI (US)

(72) Inventors: Karl Milton Taft, III, Honolulu, HI (US); Jarred Roy Engelking, Honolulu, HI (US)

(73) Assignee: PONO CORPORATION, Honolulu, HI (US)

(21) Appl. No.: 14/418,907

(22) PCT Filed: Aug. 9, 2013
(86) PCT No.: PCT/US2013/054391

§ 371 Date: Jan. 30, 2015

Related U.S. Application Data
(60) Provisional application No. 61/742,443, filed on Aug. 9, 2012.
(60) Provisional application No. 61/742,444, filed on Aug. 9, 2012.

(51) Int. Cl. A61K 47/48; A61K 31/546; A61K 33/38

(52) U.S. Cl. CPC A61K 47/48015; A61K 33/38; A61K 31/546

(57) ABSTRACT

Disclosed herein are synthesis methods for generation of conjugated anti-microbial compounds and conjugated anti-cancer compounds. Several embodiments, related to the uses of such compounds in the treatment of infections, in particular those caused by drug-resistant bacteria. Some embodiments relate to targeting cancer based on the metabolic signature of tumor cells.

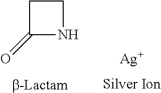

### RELATED CASES

This application claims the benefit of U.S. Provisional Application Nos. 61/742,443 and 61/742,444, both filed on Aug. 9, 2012 the entire disclosure of each of which is incorporated by reference herein.

### BACKGROUND

1. Field

Several embodiments of the invention relate generally to processes for synthesizing antimicrobial agents that are effective against targets such as gram-negative and gram-positive bacteria, gram-variable, and gram-indeterminate bacteria, including multidrug resistant strains, viruses, fungi, and other microorganisms. Additionally, several embodiments relate generally to processes for synthesizing and using novel compounds as anti-cancer agents. Methods for using the resultant compounds to treat or prevent microbial infections and/or cancer are also disclosed herein.

2. Description of Related Art

Pathogenic microbial agents include viruses, bacteria, fungi, parasites, and prions, among others and may be primary or opportunistic pathogens. Primary pathogens cause infection as a direct result of their virulence, while opportunistic pathogens typically require a compromised host defense system to produce an infection. While modern medicine has reduced the prevalence of many infections due to pathogenic microorganisms, such microorganisms continue to account for a large degree of morbidity and mortality.

### SUMMARY

Given the increasing prevalence of microorganisms that are resistant to one or more types of drugs, there is a significant need to reduce the associated morbidity and mortality related to infections by such microorganisms. To address not only infections from drug-resistant microorganisms, but also from non-resistant microorganisms, there is provided herein, in several embodiments, an anti-bacterial conjugate, comprising a targeting antibiotic and an anti-bacterial agent, wherein the anti-bacterial agent has generalized anti-bacterial activity, and wherein the anti-bacterial agent is an agent to which bacteria do not develop resistance. In several embodiments, the anti-bacterial agent is a microbicidal metal or metallic ion. In several embodiments, the metal or metallic ion is selected from the group consisting of silver, mercury, copper, iron, lead, zinc, bismuth, gold, aluminum, and combinations thereof. In one embodiment, the anti-bacterial agent is ionic silver. In additional embodiments, other metals or metallic ions may be used, including but not limited to Sc, Ti, V, Cr, Mn, Fe, Co, Ni, Cu, Zn, Ga, Rb, Sr, Y, Zr, Nb, Mo, Tc, Ru, Rh, Pd, Cd, In, Sn, Cs, Ba, Ce, Pr, Nd, Pm, Sm, Eu, Gd, Tb, Dy, Ho, Er, Tm, Yb, Lu, Hf, Ta, W, Re, Os, Jr, Pt, Ag, Au, Hg, Tl, Pb, Bi, Po, Fr, Ra, Th, Pa, U, Np, Pu, Am, Cm, Bk, Cf, Es, Fm, Md, No, Lr, Rf, Db, Sg, Bh, Hs, Mt, and combinations thereof. In several embodiments, certain of such metals are radioactive, and their dosing may be adjusted accordingly. Advantageously, in several embodiments, the radioactivity can be used as a marker of the antimicrobial effect of the administration of the conjugate.

In several embodiments, the the anti-bacterial agent is a peroxide generator. Peroxide generators may include, but are not limited to vitamin C and E. In additional embodiments, superoxide generating compounds can be used as the anti-bacterial agent. Further, the anti-bacterial agents may comprises compounds that promote or otherwise generate a localized oxidizing environment, which is damaging to the infectious microorganisms.

In several embodiments, the anti-bacterial conjugate comprises β-lactam antibiotic. In several embodiments, the use of a targeting antibiotic improves the specificity of the anti-bacterial conjugate (e.g., to reduce the risk of adverse side effects of the anti-bacterial agent on normal, non-infectious cells). In several embodiments, the β-lactam antibiotic comprises one or more of aminopenicillin, amoxicillin, ampicillin, pivampicillin, hetacillin, bacampicillin, metampicillin, talampicillin, epicillin, carboxypenicillin, carbenicillin (i.e. carindacillin), ticarcillin, temocillin, ureidopenicillins, azlocillin, piperacillin, mezlocillin, mecillinam (or pivmecillinam), sulbenicillin, methicillin, benzylpenicillin, clometocillin, benzathine benzylpenicillin, procaine benzylpenicillin, azidocillin, penamecillin, phenoxymethylpenicillin, penicillin G, penicillin V, epropicillin, benzathine phenoxymethylpenicillin, pheneticillin, cloxacillin, dicloxacillin, flucloxacillin, oxacillin, meticillin, nafcillin, faropenem, biapenem, ertapenem, antipseudomonal, doripenem, imipenem, meropenem, panipenem, cefazolin, cefacetrile, cefadroxil, cephalexin, cefaloglycin, cefalonium, cefaloridine, cefalotin, cefapirin, cefatrizine, cefazedone, cefazaflur, cefradine, cefroxadine, ceftezole, cefaclor, cefamandole, cefminox, cefonicid, ceforanide, cefotiam, cefprozil, cefbuperazone, cefuroxime, cefuzonam, cephamycin, cefoxitin, cefotetan, cefmetazole, carbacephem, loracarbef, cefixime, ceftriaxone, antipseudomonal β-lactam, ceftazidime, cefoperazone, cefcapene, cefdaloxime, cefdinir, cefditoren, cefetamet, cefmenoxime, cefodizime, cefotaxime, cefpimizole, cefpiramide, cefpodoxime, cefsulodin, cefteram, ceftibuten, ceftiolene, ceftizoxime, oxacephem, flomoxef, latamoxef, cefepime, cefozopran, cefpirome, cefquinome, ceftobiprole, ceftaroline fosamil, ceftiofur, cefquinome, cefovecin, aztreonam, tigemonam, carumonam, nocardicin A, sulbactam, tazobactam, clavam, clavulanic acid, imipenem, cilastatin, and sultamicillin.

In several embodiments, the β-lactam antibiotic comprises one or more of cefazolin, a cefotaxime derivative, a cephalothin derivative, a tetracycline derivative, a ceftriaxone derivative, and an aztreonam derivative (e.g., the anti-bacterial conjugate may, in some embodiments, comprise a mixture of various targeting antibodies). In additional embodiments, non-β-lactam antibiotics are used as the targeting antibodies.

Furthermore, there is provided herein a method of treating or preventing a bacterial infection in a subject, comprising identifying a subject suffering from a bacterial infection or in need of bacterial infection prophylaxis; and delivering an anti-bacterial conjugate according to any of the embodiments disclosed herein, thereby treating the infection.

In several embodiments, anti-bacterial conjugate is delivered to the subject topically, subcutaneously, nasally, intraarterially, intramuscularly, intracranially, by intraosseous infusion, intrathecally, intraperitoneally, intravesically, intravitreally, intracavernously, intravaginally, transdermally, transmucosally, orally, anally, or intravenously.

Also provided herein is a use of a composition comprising an anti-microbial targeting moiety complexed to an anti-microbial effector moiety for the treatment of a microbial infection. In several embodiments, the anti-microbial targeting moiety comprises an antibiotic, such as, for example a beta-lactam antibiotic or the backbone of a beta-lactam antibiotic. In several embodiments, the anti-microbial effector moiety comprises a silver ion. In additional embodiments, the anti-microbial effector moiety comprises a compound that generates free radicals, or may also comprise a compound that generates peroxide. In several embodiments, the microbial infection is due to drug-resistant microorganisms. For example, the drug-resistant microorganisms may be one or more of a drug resistant gram negative bacterium and a drug resistant gram positive bacterium. In several embodiments, the drug-resistant microorganisms are one or more of carbapenem-resistant enterobacteriaceae (CRE) and methicillin-resistant staphylococcus aureus (MRSA).

In addition to infections (including drug-resistant infections), cancer continues to be a major source of morbidity and mortality around the world. Therefore, there are provided, in several embodiments, anti-cancer conjugates, comprising a nutrient-based targeting agent and an anti-cancer agent. In several embodiments, the anti-cancer agent is a cytotoxic metal or metal ion selected from the group consisting of silver mercury, copper, iron, lead, zinc, bismuth, gold, and aluminum. In one embodiment, the anti-cancer agent is ionic silver. In additional embodiments, other cytotoxic metals or metal ions may be used, alone or in combination with those listed above. For example, in several embodiments, the anti-cancer agent is a cytotoxic metal or metal ion comprising one or more of Sc, Ti, V, Cr, Mn, Fe, Co, Ni, Cu, Zn, Ga, Rb, Sr, Y Zr, Nb, Mo, Tc, Ru, Rh, Pd, Cd, In, Sn, Cs, Ba, Ce, Pr, Nd, Pm, Sm, Eu, Gd, Tb, Dy, Ho, Er, Tm, Yb, Lu, Hf, Ta, W, Re, Os, Jr, Pt, Ag, Au, Hg, Tl, Pb, Bi, Po, Fr, Ra, Th, Pa, U Np, Pu, Am, Cm, Bk, Cf, Es, Fm, Md, No, Lr, Rf, Db, Sg, Bh, Hs, Mt, and combinations thereof. Advantageously, several of these ions are radioactive, and therefore can provide anti-cancer effects via multiple mechanisms of action. For example, in several embodiments, the use of a radioactive metal ion results in synergistic anti-cancer effects, as the metal ion not only can disrupt the cancer cells because of the effects of the metal on cellular respiration etc., but can also deliver radioactivity to the cancer cell, thereby inducing DNA damage in the cancer cell.

Additionally, in several embodiments, the anti-cancer agent is a peroxide generator. Suitable peroxide generators include, but are not limited to, vitamins C and E. In additional embodiments, superoxide generating compounds can be used as the anti-cancer agent. Further, the anti-cancer agents may comprise compounds that promote or otherwise generate a localized oxidizing environment, which is damaging to the tumor cells.

In several embodiments, the nutrient-based targeting agent is selected from the group consisting of fructose, glucose, galactose, sucrose, maltose, lactose, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, glucosamine, monosaccharides, dissaccharides, trisaccharides, oligosaccharides, polysaccharides, dipeptides, oligopeptides, polypeptides, proteins, and combinations thereof. In several embodiments, nutrient-based targeting moiety is an energy component selected from the group consisting of fructose, glucose, glutamine, glucosamine, among others, and amino-acid-based moieties. Additionally, in several embodiments, the nutrient-based targeting moiety is a functionalized derivative. Advantageously, the use of a nutrient-based or energy-based targeting component capitalizes on the elevated metabolism of tumor cells as compared to normal cells. Because of their elevated metabolism, the tumor cells have energy requirements that exceed those of normal cells, and thus, the tumor cells will take up the nutrient-based or energy-based targeting component to a greater degree than the normal cells. This provides a “metabolic targeting” that helps to reduce the chances of deleterious side effects in normal cells.

There are also provided herein methods of treating a cancer in a subject, comprising identifying a subject suffering from cancer and delivering an anti-cancer conjugate to the subject. In several embodiments, the wherein the anti-cancer conjugate is delivered to the subject topically, subcutaneously, nasally, intraarterially, intramuscularly, intracranially, by intraosseous infusion, intrathecally, intraperitonieally, intravesically, intravitreally, intracavernously, intravaginally, transdermally, transmucosally, orally, anally, or intravenously.

Also provided herein is the use of a composition comprising an anti-cancer targeting moiety complexed to an anti-cancer effector moiety for the treatment of a cancer. In several embodiments, the anti-cancer targeting moiety comprises a nutritional energy source capable of metabolism by the cancer. For example, in several embodiments the anti-cancer targeting moiety is selected from the group consisting of fructose, glucose, galactose, sucrose, maltose, lactose, alanine arginine, asparagine, aspartic acid, cysteine, glutamine, glutamic acid, glycine, histidine, isoleucine, leucine, lysine, methionine, phenylalanine, proline, serine, threonine, tryptophan, tyrosine, glucosamine, monosaccharides, dissaccharides, trisaccharides, oligosaccharides, polysaccharides, dipeptides, oligopeptides, polypeptides, proteins, and combinations thereof. In one embodiment the anti-cancer effector moiety comprises a silver ion. In one embodiment the anti-cancer effector moiety comprises a compound that generates free radicals (e.g., superoxide), while in an additional embodiment the anti-cancer effector moiety comprises a compound that generates peroxide.

The methods summarized above and set forth in further detail below describe certain actions taken by a practitioner; however, it should be understood that they can also include the instruction of those actions by another party. Thus, actions such as “administering a silver-complexed antibiotic” include “instructing the administration of a silver-complexed antibiotic.”

### BRIEF DESCRIPTION OF THE DRAWINGS

FIG. 1 is an illustration that depicts the chemical structure of the most basic, undecorated beta-lactam core and ionic silver.

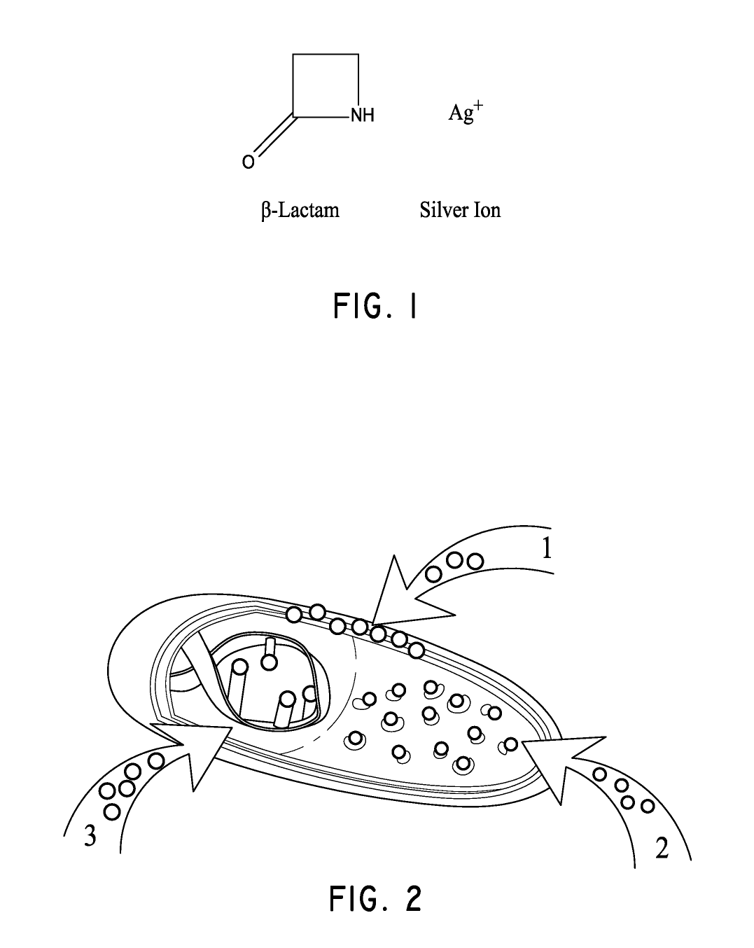

FIG. 2 is an illustration that depicts silver ion anti-bacterium pathways.

FIGS. 3A-3I depict embodiments of silver ion compounds according to several embodiments disclosed herein.

FIG. 4 is an illustration of a synthetic pathway for silver ion containing Cefotaxime Derivative.

FIGS. 5A-5C is an illustration of novel compound embodiments with an ionic silver warhead or possible peroxide generator warhead.

FIGS. 6A-6D is an illustration that depicts the chemical structure of four preferred embodiments of the conjugates.

FIG. 7 is an illustration that depicts one embodiment of a conjugate synthesis pathway.

FIG. 8 is an illustration that depicts an additional embodiment of a conjugate synthesis pathway.

FIG. 9 is an illustration that depicts an additional embodiment of a conjugate synthesis pathway.

Those of skill in the art understand that the drawings, described herein, are for illustrative purposes only. The drawings are not intended to limit the scope of the present teachings in any way.

### DETAILED DESCRIPTION

In the following description, numerous specific details are set forth in order to provide a thorough understanding of the present invention. It will be apparent, however, to one skilled in the art that the present invention is practiced without limitation to some or all of these specific details. In other instances, well-known process steps have not been described in detail in order to not unnecessarily obscure the invention.

As used herein, a the term “subject” shall be given its ordinary meaning and shall also include any organism, including an animal, for which diagnosis, screening, monitoring or treatment is contemplated. Animals include mammals such as primates and domesticated animals. In several embodiments, the primate is a human. A patient refers to a subject such as a mammal, primate, human or livestock subject afflicted with a disease condition or for which a disease condition is to be determined or risk of a disease condition is to be determined.

As used herein the term “inhibit” shall be given its ordinary meaning and shall not be interpreted to require absolute inhibition. Similarly, the term “prevent” does not require absolute prevention. Inhibiting the growth or activity of a microorganism shall also refer to treating an infection caused by microorganisms. Inhibiting the growth or activity of a microorganism shall include reducing its growth, activity and/or viability by a measurable amount, for example at least 5%, at least 10%, at least 20%, at least 30%, at least 40%, at least 50%, at least 60%, at least 70%, at least 80%, at least 85%, at least 90%, at least 95%, at least 99%, or 100%. Likewise, the growth of a tumor or enhancing the regression of a tumor includes reducing the size of an existing tumor. Preventing the growth of a tumor includes preventing the development of a primary tumor or preventing further growth of an existing tumor. Reducing the size of a tumor includes reducing the size of a tumor by a measurable amount, for example at least 5%, at least 10%, at least 20%, at least 30%, at least 40%, at least 50%, at least 60%, at least 70%, at least 80%, at least 85%, at least 90%, at least 95%, at least 99%, or 100%.

### Anti-Microbial Compounds

As used herein, the term “microorganism” shall be given its ordinary meaning and shall include, but not be limited to, viruses (including but not limited to human immunodeficiency virus, herpes simplex virus, papilloma virus, parainfluenza virus, influenza viruses including H1N1, EBV, CMV, hepatitis A, B, C, D, E, F, and G, Coxsackie Virus, herpes zoster, measles, mumps, rubella, rabies, West Nile, pneumonia, hemorrhagic viral fevers, and the like), JC virus, HTLV, prions, parasites, fungi, mold, yeast and bacteria (both gram-positive, gram-negative, gram-variable, and gram-indeterminate including acid-fast bacilli) including, among others, Candida including C. albicans, Aspergillus niger, Escherichia coli (E. coli), Klebsiella, Pseudomonas aeruginosa (P. aeruginosa), and Staphylococcus including S. aureus, Group A and other streptococci including S. pneumoniae, Mycobacterium including M. tuberculosis and Mycobacterium avium-intracellulare, Campylobacter jejuni, Salmonella, Shigella, Bacillus including anthracis, Borrelia, Rickettsia, Pneumocystis carinii, and a variety of drug resistant organisms including bacteria. The terms microorganism and microbe shall be used interchangeably. Microbes can include wild-type, genetically-engineered or modified organisms. The term shall also include those microogansims that exhibit partial or complete drug resistance, such as, for example, the gram negative bacterium carbapenem-resistant enterobacteriaceae (CRE), extended spectrum beta-lactamase-producing bacteria (ESBL), or the drug resistant gram positive bacterium, methicillin-resistant staphylococcus aureus (MRSA), vancomycin-resistant staphylococcus aureus (VRSA), or vancomycin-resistant enterococcus (VRE).

The prevalence of antibiotic and/or drug resistance in bacteria is becoming one of the leading public health threats. Current antibiotics interfere with the critical biological processes of the pathogens and cause death or growth arrest of the bacteria. As a result, antibiotic therapy exerts a strong selective pressure to favor emergence of antibiotic resistant strains. For that reason, the number of bacteria strains that are resistant to front-line antibiotics is growing at an alarming rate, yet there are no signs of replacement treatments in the market or pipeline. The few alternatives that do exist are either expensive, highly toxic, and/or slow acting. Resistance is even growing among infections that today are considered easily treatable, such as tuberculosis, salmonella, E. coli, and gonorrhea.

Resistant pathogens are especially prevalent in the one place where people are supposed to be out of harm's way: hospitals. Especially dangerous strains such as methicillin-resistant Staphylococcus aureus (MRSA) are practically bred in hospitals, with healthcare-associated infections on the rise. 2% of Staphylococcus aureus infections in US intensive-care units were MRSA in 1974, 22% in 1995, and 64% in 2004 (Klevens R M et al. Clinical Infectious Diseases 2006, 42, 389-391).

Separately, infections caused by certain microorganisms, such as certain gram-negative bacteria are generally unresponsive to present-day antibiotics due to the inability for medicines to penetrate their thicker cell walls. Certain acid-fast bacilli including Mycobacterium tuberculosis have also become multi-drug resistant. This market is extremely under addressed, while cases of gram-negative bacterial infections are on the rise. Thus, there is a dire need for novel approaches to combat resistant and unresponsive microorganisms, including but not limited to bacteria, viruses, fungi, and parasites (e.g., malaria). Several embodiments of the compositions and methods disclosed herein address that need.

### Microbial Targeting Moieties

In several embodiments, a targeted anti-microbial agent is provided. In several embodiments, a targeting moiety is functionally linked to the anti-microbial agent to provide the targeted anti-microbial agent. In several embodiments, the targeting moiety is a β-lactam antibiotic or β-lactam antibiotic backbone. Bacteria constantly remodel their cell walls, simultaneously building and breaking down portions as they grow and divide. β-lactams can be used as targeting moieties because β-lactams bind to enzymes that link polymeric molecules in the cell wall of a bacterium. In several embodiments, the targeting β-lactam is part of an antibiotic (e.g., a β-lactam antibiotic).

In several embodiments, the β-lactam antibiotic is selected from one of the following classes of β-lactam antibiotics: penicillin derivatives, cephalosporins, cephems, monobactams, carbapenems, cephamycins, monobactams, and beta-lactamase inhibitors. In several embodiments, the β-lactam antibiotic used for targeting the bacterium includes one or more of cefazolin, cefotaxime, cephalothin, tetracycline, ceftriaxone, and aztreonam, and their derivatives. In several embodiments, the β-lactam antibiotic used for targeting the bacterium includes one or more of aminopenicillin, amoxicillin, ampicillin, pivampicillin, hetacillin, bacampicillin, metampicillin, talampicillin, epicillin, carboxypenicillin, carbenicillin (i.e. carindacillin), ticarcillin, temocillin, ureidopenicillins, azlocillin, piperacillin, mezlocillin, mecillinam (or pivmecillinam), sulbenicillin, methicillin, benzylpenicillin, clometocillin, benzathine benzylpenicillin, procaine benzylpenicillin, azidocillin, penamecillin, phenoxymethylpenicillin, penicillin G, penicillin V, epropicillin, benzathine phenoxymethylpenicillin, pheneticillin, cloxacillin, dicloxacillin, flucloxacillin, oxacillin, meticillin, nafcillin, faropenem, biapenem, ertapenem, antipseudomonal, doripenem, imipenem, meropenem, panipenem, cefazolin, cefacetrile, cefadroxil, cephalexin, cefaloglycin, cefalonium, cefaloridine, cefalotin, cefapirin, cefatrizine, cefazedone, cefazaflur, cefradine, cefroxadine, ceftezole, cefaclor, cefamandole, cefminox, cefonicid, ceforanide, cefotiam, cefprozil, cefbuperazone, cefuroxime, cefuzonam, cephamycin, cefoxitin, cefotetan, cefmetazole, carbacephem, loracarbef, cefixime, ceftriaxone, antipseudomonal penicillins and cephalosporins, ceftazidime, cefoperazone, cefcapene, cefdaloxime, cefdinir, cefditoren, cefetamet, cefmenoxime, cefodizime, cefotaxime, cefpimizole, cefpiramide, cefpodoxime, cefsulodin, cefteram, ceftibuten, ceftiolene, ceftizoxime, oxacephem, flomoxef, latamoxef, cefepime, cefozopran, cefpirome, cefquinome, ceftobiprole, ceftaroline fosamil, ceftiofur, cefquinome, cefovecin, aztreonam, tigemonam, carumonam, nocardicin A, sulbactam, tazobactam, clavam, clavulanic acid, imipenem, cilastatin, sultamicillin, and/or any combination thereof. In several embodiments, the β-lactam antibiotic used for targeting the bacterium can include derivatives of any of the above β-lactam, β-lactamase inhibitor, and other antibiotics or components thereof. β-lactam antibiotics can mute the bacteria's response to attack by inhibiting penicillin binding proteins (PBPs), which are essential for the bacterial cell wall biogenesis. In several embodiments, the β-lactams also inhibit the formation of cross-links in the bacterial cell wall. In several embodiments, this cross-linking inhibition weakens the cell wall of the bacterium and eventually leads to cytolysis or death due to osmotic pressure. In addition, in several embodiments, the build-up of cell wall precursors triggers the activation of enzymes that digest the bacteria's existing cell scaffold. This imbalance between cell wall production and degradation is responsible for the rapid cell-killing action, even in the absence of cell division. Thus, in some embodiments, the β-lactam antibiotic targets and kills microbes. In several embodiments, the ability of the β-lactam antibiotic to target and/or kill microorganisms is synergistically enhanced by its being complexed to a metal ion (e.g., a silver ion).

In several embodiments, other antibiotics can be used instead of or in addition to β-lactam antibiotics. Other antibiotics that target the bacterial cell wall (e.g., other penicillins and/or cephalosporins) are used in some embodiments. In some embodiments, those antibiotics that target the cell membrane (e.g., polymixins) are used. In some embodiments, those antibiotics that interfere with essential bacterial enzymes (e.g., rifamycins, lipiarmycins, quinolones, and sulfonamides) are used. Those that target protein synthesis (e.g., macrolides, lincosamides and tetracyclines) are also used in several embodiments. The antibiotics used may be considered “narrow” or “broad” spectrum. The antibiotics may also include, in several embodiments, cyclic lipopeptides (e.g., daptomycin), glycylcyclines (e.g., tigecycline), oxazolidinones (e.g., linezolid) and/or lipiarmycins (e.g., fidaxomicin). In several embodiments, one or more of amikacin, gentamicin, kanamycin, neomycin, netilimicin, tobramycin, paromomycin, spectinomycin, geldanamycin, herbimycin, streptomycin, cephalexin, teicoplanin, vancomycin, telavancin, clindamycin, lincomycin, daptomycin, azithromycin, clarithromycin, dirithromycin, erythromycin, roxithromycin, troleandomycin, telithromycin, spiramycin, furazolidone, nitrofurantoin, linezolid, posizolid, radezolid, torezolid, bacitracin, colistin, polymyxin B, ciprofloxacin, enoxacin, gatifloxacin, levofloxacin, lomefloxacin, moxifloxacin, naldixic acid, norfloxacin, ofloxacin, trovafloxacin, grepafloxacin, sparfloxacin, temafloxacin, mafenide, sulfacetamide, sulfadiazine, sulfadimethoxine, sulfamethizole, sulfamethoxazole, sulfanilamide, sulfasalazine, sulfisoxazole, trimethoprim-sulfamethoxazole, sulfonamidochrysoidine, demeclocycline, doxycycline, minocycline, oxytetracycline, tetracycline, clofazimine, dapsone, capreomycin, cycloserine, ethambutol, ethionamide, isoniazid, pyrazinamide, rifampicin, rifabutin, rifapentine, streptomycin, arsphenamine, chloramphenicol, fosfomycin, fusidic acid, metronidazole, mupirocin, platensimycin, quinupristin, dalfopristin, thiamphenicol, tigecycline, tinidazole, trimethoprim, and/or combinations thereof. Combinations of one or more class of antibacterials are used in some embodiments.

Several of the above-listed antibiotics function by targeting bacterial cell walls (as in the β-lactams), or by other mechanisms. For instance, in several embodiments, the antibiotics function by targeting the cell membrane of bacteria. In several embodiments, the above antibiotics function by interfering with essential bacterial enzymes to kill the bacteria. In several embodiments, the antibiotics target essential bacterial protein syntheses. Thus, depending on the embodiment, the targeting moiety may exert anti-microbial effects on its own (e.g., in treatment of non-drug resistant or low-drug resistant infections) or may serve primarily as a targeting agent (e.g., in treatment of substantial or wholly-drug resistant infections).

In some embodiments, the targeted antimicrobial compound has antiviral activity, including but not limited to Abacavir, Acyclovir, Adefovir, Amprenavir, Atazanavir, Cidofovir, Darunavir, Delavirdine, Didanosine, Docosanol, Efavirenz, Elvitegravir, Emtricitabine, Enfuvirtide, Etravirine, Famciclovir, Foscarnet, Fomivirsen, Ganciclovir, Indinavir, Idoxuridine, Lamivudine, Lopinavir Maraviroc, MK-2048, Nelfmavir, Nevirapine, Penciclovir, Raltegravir, Rilpivirine, Ritonavir, Saquinavir, Stavudine, Tenofovir Trifluridine, Valaciclovir, Valganciclovir, Vidarabine, Ibacitabine, Amantadine, Oseltamivir, Rimantidine, Tipranavir, Zalcitabine, Zanamivir, and Zidovudine, and derivatives and analogs thereof.

In some embodiments, the targeted antimicrobial compound has antifungal activity, including but not limited to Fluconazole, Isavuconazole, Itraconazole, Ketoconazole, Miconazole, Clortrimazole, Voriconazole, Posaconazole, Ravuconazole, natamycin, lucensomycin, nystatin, amphotericin B, echinocandins, Cancidas, pradimicins, beanomicins, nikkomycins, sordarins, allylamines, Triclosan, Piroctone, phenpropimorph, terbinafine, antifungal peptide, and derivatives and analogs thereof.

In some embodiments, the targeted antimicrobial compound has antihelminthic activity, including but not limited to thiabendazole, mebendazole, albendazole, quinacrine hydrochloride, niclosamide, pyrantel pamoate, tetramisole, levamisole, bephenium, and praziquantel, and derivatives and analogs thereof.

In some embodiments, the targeted antimicrobial compound has antiprotozal and antiparastic activity, including but not limited to atovaquone, chloroquine phosphate, quinacrine hydrochloride, iodoquinol, pyrimethamine, and mefloquine hydrochloride, and derivatives and analogs thereof. In some embodiments, conjugates as described herein can be used, for example, to treat conditions including but not limited to pneumonia, salmonellosis, meningitis or other CNS infection, endocarditis, osteomyelitis, urinary tract infection, pyelonephritis, toxic shock syndrome, pharyngitis, infections endometriosis, diphtheria, septicemia, gastroenteritis, urinary tract infections, otitis media, salmonellosis, shigellosis, tuberculosis, staphylodermatitis, keratitis, impetigo, cellulitis, erysipelas, abscesses including spinal epidural abscess, or endophthalmitis.

In several embodiments, one or more of the above antibiotics may be used in combination to kill and/or target microbes. In several embodiments, two, three, four, five, or six or more of the above antibiotics can be used in combination as an antimicrobial.

In additional embodiments, the anti-microbial targeting moiety comprises a nutritional source for bacteria (or other microorganisms) which would be taken up by the microorganism in the normal course of its metabolism. Upon taking up the nutrient, which is complexed to an anti-microbial effector moiety, the effector moiety inhibits the growth or activity of the microorganism. Advantageously, in many cases, cells causing an infection have a more rapid metabolism, and thus may preferentially take up the complexed anti-microbial composition. Growth media used in research provide numerous examples of carriers for the anti-microbial targeting moiety. In several embodiments, the carbon, nitrogen, oxygen, and sulfur compounds are defined food sources for the bacteria. For example, in several embodiments, the nutrient-based antimicrobial targeting moiety is selected from the group consisting of fructose, glucose, galactose, sucrose, maltose, lactose, alanine arginine, asparagine, aspartic acid, cysteine, glutamine, glutamic acid, glycine, histidine, isoleucine, leucine, lysine, methionine, phenylalanine, proline, serine, threonine, tryptophan, tyrosine, glucosamine, monosaccharides, dis saccharides, trisaccharides, oligosaccharides, polysaccharides, dipeptides, oligopeptides, polypeptides, proteins, and any combination thereof. In several embodiments, tryptone is used as the anti-microbial targeting moiety. In several embodiments, yeast extract is used as the anti-microbial targeting moiety. In several embodiments, functionalized derivatives of any of these species may be employed. In several embodiments, vitamins and fatty acids may be used to target the microorganisms.

### Anti-Microbial Effector Moieties

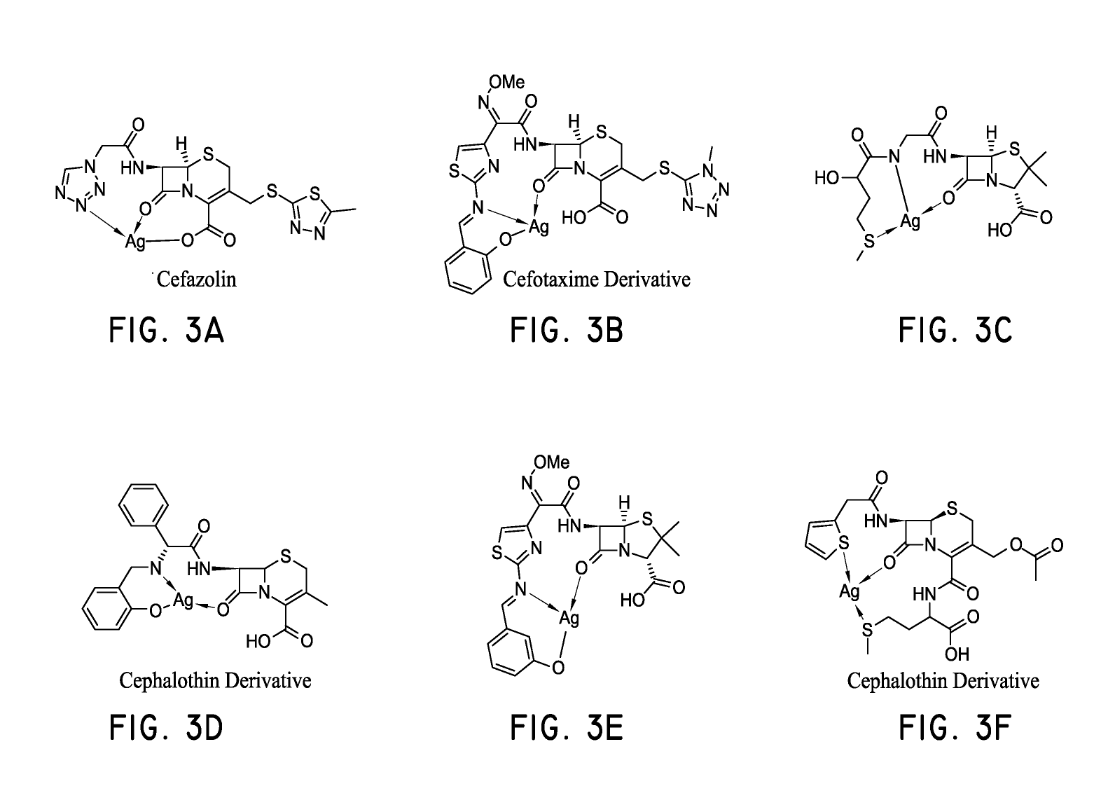

β-lactam antibiotics work by attacking a bacterium in a specific fashion (for example, disrupting cell wall synthesis). Because the killing is done precisely, bacteria may develop mutations that confer resistance toward β-lactam antibiotics. Unlike antibiotics, certain metal ions (e.g., silver ions) simultaneously attack many sites in bacterium which stops reproduction and/or causes bacterial death. In contrast to antibiotics, silver ions kill microbes in a broad, unspecific fashion, equivalent to “tossing a bomb” at a bacterium. For instance, and not to be limited by theory, silver ions can be used to attack the bacterium's entire respiratory system (1), metabolism (2), and/or cell division or DNA (3) as illustrated in FIG. 2. At the plasma membrane, silver binds either to membrane bound proteins or the lipid bilayer itself and destabilizes the membrane, causing ion leakage and cell rupture. Inside the cell, silver binds to and disrupts the function of mitochondrial membranes, interfering with the energy (ATP) yielding reactions of the respiratory chain. Silver can also bind specifically to cellular enzymes and DNA, thus interfering with their functions. There are no known silver-resistant medically-relevant strains of bacteria. Thus, several embodiments of the present invention advantageously capitalize on the mechanisms of action of silver ions (or other metal ions), alone or in combination with the antibiotic to which they are coupled, to provide unexpectedly efficacious antimicrobial effects. In addition to bacterial, silver can also have other antimicrobial effects, including antiviral, antifungal, and antiparasitic effects for example.

Ionic silver (Ag⁺) is extremely toxic to a broad variety of organisms including bacteria. For example, silver has toxicity in both gram-positive and gram-negative forms of bacteria. Ionic silver has shown strong biocidal efficacy against at least sixteen additional species of bacteria to-date, including mycobacterium tuberculosis. The multimodal efficacy of ionic silver (or other metal ions) occurs at very low concentrations making it much more difficult for silver resistance to develop. Moreover, silver ions, even in substantial concentrations, are not typically known to pose any significant harm to humans and have shown effectiveness against a number of microorganisms including both gram-positive and gram-negative bacteria.

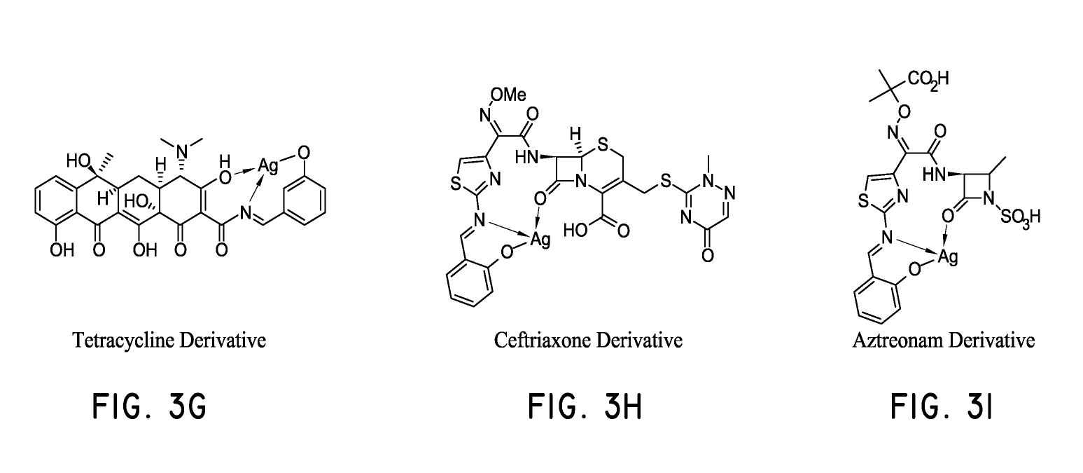

In several embodiments, ionic silver is complexed to a β-lactam antibiotic to kill bacteria. In several embodiments, the β-lactam functionality of a β-lactam antibiotic binds to ionic silver. In several embodiments, the β-lactam moiety can target bacteria and deliver ionic silver to the bacteria. In several embodiments, upon delivery by the β-lactam antibiotic, the ionic silver disrupts bacteria through any one of the above mechanisms (or multiple of the above-referenced mechanisms). In several embodiments, a synergistic effect is achieved using a silver ion complex with β-lactam antibiotic, because both of these components have toxicity in bacteria. In several embodiments, the β-lactam antibiotic improves efficacy of ionic silver by targeting the cell wall of the bacteria and directing the ionic silver to the bacteria. As mentioned above, other antibiotics (e.g., non-β-lactam antibiotics may also be used). FIGS. 3A-3I depict several non-limiting embodiments of silver-ion containing anti-microbial compounds.

In several embodiments, other microbicidal metals may be used in combination with the β-lactam antibiotic. For instance, in several embodiments, the β-lactam antibiotic is complexed to ions of silver, mercury, copper, iron, lead, zinc, bismuth, gold, aluminum, or other metals. In several embodiments, Sc, Ti, V, Cr, Mn, Fe, Co, Ni, Cu, Zn, Ga, Rb, Sr, Y Zr, Nb, Mo, Tc, Ru, Rh, Pd, Cd, In, Sn, Cs, Ba, Ce, Pr, Nd, Pm, Sm, Eu, Gd, Tb, Dy, Ho, Er, Tm, Yb, Lu, Hf, Ta, W, Re, Os, Ir, Pt, Ag, Au, Hg, Tl, Pb, Bi, Po, Fr, Ra, Th, Pa, U Np, Pu, Am, Cm, Bk, Cf, Es, Fm, Md, No, Lr, Rf, Db, Sg, Bh, Hs, Mt, and/or combinations thereof may be used in combination with one or more β-lactam (or other type) antibiotics to kill bacteria. In several embodiments, combinations of one or more β-lactam antibiotics and one or more of the above metal (in their ionic form) may be used as anti-microbial agents. In several embodiments, ionic silver and/or any other anti-microbial metal ion may be complexed to any of the above non-β-lactam antibiotics for use as an anti-microbial. In some embodiments, at least 1, 2, 3, 4, 5, or more anti-microbial metal ions complex, e.g., at different sites, to a single targeting moiety. In some embodiments, the ability to complex a plurality of anti-microbial metal ions to a single targeting moiety can advantageously improve efficacy by increasing the concentration of anti-microbial metal ions reaching the target location. In some embodiments, complexing a microbicidal metal or metal ion to an antibiotic targeting moiety can unexpectedly and advantageously decrease the mean inhibitory concentration (MIC) required for a particular microbe, such as a bacteria, by a factor of 2, 4, 8, 16, 32, or more with respect to the MIC of the antibiotic targeting moiety alone without the microbicidal metal or metal ion.

In several embodiments, rather than complexing a targeting moiety, such as a β-lactam antibiotic, to a metal ion, other anti-microbial moieties are used, either in place of, or in addition to a metal ion. For example, those molecules that can lead to generation of reactive oxygen species can be complexed to an antibiotic and, upon administration to an individual with an infection (either drug resistant or non-resistant) generate localized reactive oxygen species, thus leading to inhibition and/or death of the infectious microorganisms. For example, in several embodiments, compounds that promote the formation of superoxide ions are complexed to an anti-microbial targeting moiety. In several embodiments, generation of superoxide ions leads to one or more of DNA damage, mitochondrial dysfunction, increased apoptosis, each of which can occur in combination with any of the others, ultimately resulting in anti-microbial effects. In several embodiments, pro-oxidant compounds, such as for example vitamin C, zinc, vitamin E, and/or polyphenol antioxidants are used. It is surprising, in several embodiments, that molecules that are typically associated with antioxidant effects can be employed in a specifically targeted pro-oxidant capacity. However, for example, vitamin C (via the Fenton reaction) can reduce metal ions and result in the generation of free radicals, leading to antimicrobial effects. Thus, in several embodiments, a composition comprising a mixture of targeting moieties complexed to metal ions and targeting moieties complexed to vitamin C result in synergistic antibacterial effects. In additional embodiments, molecules that generate intracellular peroxides are complexed to anti-microbial targeting moieties. These molecules include those having one or more hydroxyl groups. In some embodiments, peroxide generating molecules that are known to be toxic to cells in other biological contexts are used to yield antimicrobial effects. For example, in several embodiments, one or more of pyocyanin (1-hydroxy-N-methylphenazine), dopamine, 6-hydroxydopamine, 6-aminodopamine, 6,7-dihydroxytryptamine, and dialuric acid are complexed to a targeting moiety. In several embodiments, prooxidant proteins are coupled to a targeting moiety. For example, in several embodiments, a member of the metallothionein family is complexed and results in production of hydroxyl radicals upon administration. In several embodiments, flavonoids including, but not limited to, flavones, isoflavones, and/or flavanones serve as prooxidants (particularly in the presence of copper ions) and are complexed to anti-microbial targeting moieties. In several embodiments, delivery of catechins, e.g., epigallocatechin or epicatechin generates hydrogen peroxide and/or hydroxyl radical that yield anti-microbial effects. In several embodiments, esters of fumaric acid (e.g., dimethyl fumarate or methylhydrogenfumarate) are complexed to a targeting moiety. In several embodiments, a peroxide generator, superoxide promoter, etc. is employed in conjunction with a β-lactam antibiotic instead of, or in addition to, a microbicidal metal ion.

### Synthesis of Anti-Microbial Compounds

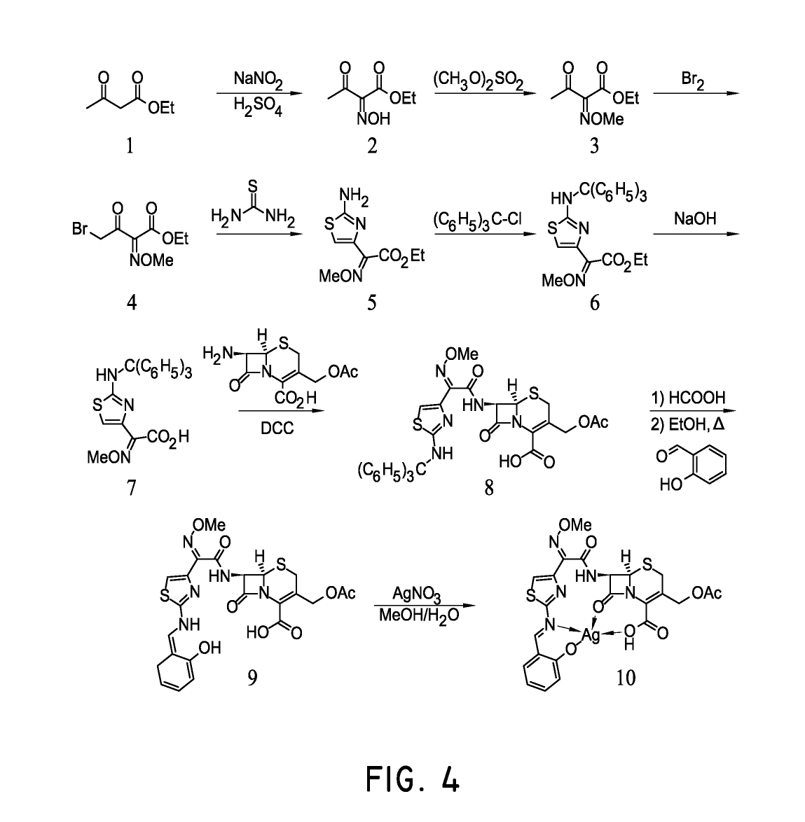

According to several embodiments, synthesis schemes of anti-microbial compounds are provided. The core moieties of β-lactam antibiotics (including but not limited to those penicillins, e.g., ampicillin, and cephalosporins) are readily available from commercial sources in the form of (+)-6-aminopenicillanic acid (6-APA) and 7-aminocephalosporanic acid (7-ACA). In several embodiments, the free amine off of the beta-lactam ring is coupled with an activating agent through a substitution or coupling reaction. The resulting parent compound is then complexed with silver (or other metal ions or anti-microbial effectors) to provide the active target molecule. Discussed in detail below is a non-limiting example of one such scheme. Other schemes (e.g., those involving other antibiotic backbones and/or other metal ions are also contemplated and within the scope of the present disclosure. In one embodiment, shown as a non-limiting example in FIG. 4, (6R,7R)-3-(acetoxymethyl)-7-((Z)-2-(2-(((Z)-(2-hydroxycyclohexa-2,4-dien-1-ylidene)methyl)amino)thiazol-4-yl)-2-(methoxyimino)acetamido)-8-oxo-5-thia-1-azabicyclo[4.2.0]oct-2-ene-2-carboxylic acid silver complex (10), is synthesized by nitrosation of acetoacetic ester (1) with nitrous acid which produces isonitrosoacetoacetic ester (2). O-Methylation of the hydroxyl group of the obtained product with dimethylsulfate in the presence of potassium carbonate provides ethyl 2-(methoxyimino)acetoacetate (3). Brominating the resulting product with bromine in methylene chloride in the presence of p-toluenesulfonic acid provides 4-bromo-2-methoxyiminoacetoacetic ester (4). Reacting 4 with thiourea according to the classic scheme of preparing of thiazoles from α-bromocarbonyl compounds and thioamides provides the ethyl ester of 2-(2-amino-4-thiazolyl)-2-methoxyiminoacetic acid (5). Reacting 5 with triphenylchloromethane in the presence of triethylamine results in a trityl protection of the amino group, forming the ethyl ester of 2-(2-tritylamino-4-thiazolyl)-2-methoxyminoacetic acid (6), which is hydrolyzed to the acid (7) using sodium hydroxide. The resulting acid 7 is used for acylating of 7-aminocephalosporanide acid in the presence of dicyclohexylcarbodiimide (DCC), giving tritylated cefotaxime, α-O-methyloxime acetate 7-[2-(2-tritylamino)-4-thiazolyl-glycoxylamido]-3-(hydroxymethyl)-8-oxo-5-thia-1-azabicyclo[4.2.0]oct-2-en-2-carboxylic acid (8). Finally, removing the trityl protection from the synthesized product (8) using dilute formic acid gives cefotaxime. [Synthesis of Essential Drugs, ISBN: 978-0-444-52166-8]. Cefotaxime is coupled with 2-hydroxybenzaldehyde to provide (6R,7R)-3-(acetoxymethyl)-7-((Z)-2-(2-(((Z)-(2-hydroxycyclohexa-2,4-dien-1-ylidene)methyl)amino)thiazol-4-yl)-2-(methoxyimino)acetamido)-8-oxo-5-thia-1-azabicyclo[4.2.0]oct-2-ene-2-carboxylic acid (9). The resulting acid 9 is complexed with silver nitrate to provide (6R,7R)-3-(acetoxymethyl)-7-((Z)-2-(2-(((Z)-(2-hydroxycyclohexa-2,4-dien-1-ylidene)methyl)amino)thiazol-4-yl)-2-(methoxyimino)acetamido)-8-oxo-5-thia-1-azabicyclo[4.2.0]oct-2-ene-2-carboxylic acid silver complex (10) or cefotaxime derivative silver complex. The exact silver coordination may vary, depending on the embodiment.

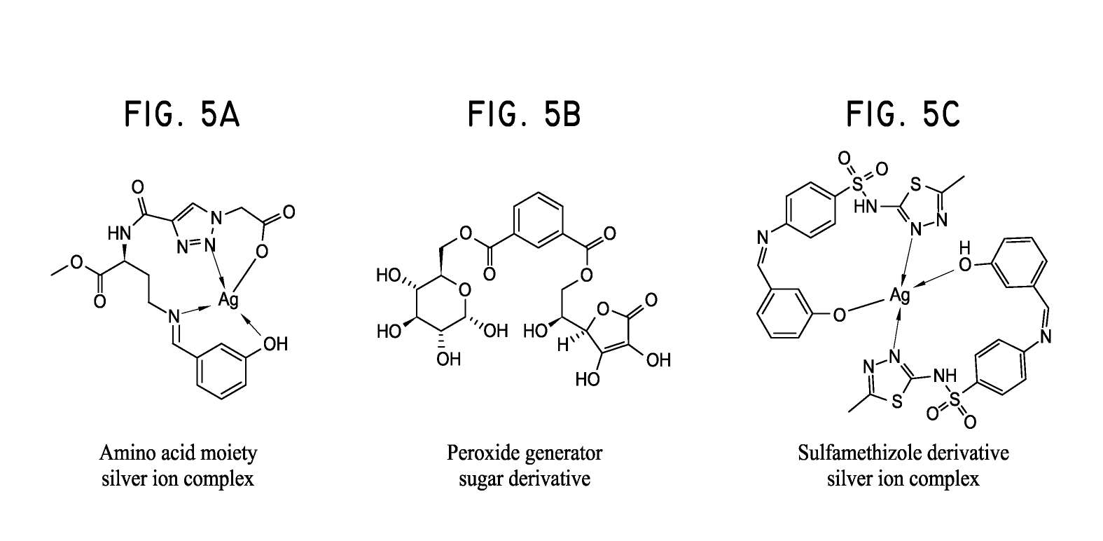

Additional non-β-lactam containing embodiments shown in FIGS. 5A-5C and are in no way meant to be limiting. These include, for example, an amino acid moiety complexed with silver ion (5A), a peroxide generator sugar derivative (5B), and antibiotic derivative complexed with silver ion (5C).

### Treatment of Microbial Infections

The treatment of microbial infections using the compositions disclosed herein can be achieved in a variety of ways, depending on the embodiment. Doses of the compositions that employ an antibiotic anti-microbial targeting moiety can mirror doses of antibiotics that are established in the medical field. For example, if a β-lactam antibiotic is used as the anti-microbial targeting moiety, doses may range from about 100 mg to about 8 g per administration, about 100 mg to about 4 g, about 250 mg to about 4 g, about 1 g to about 2 g, about 2 g to about 4 g, about 250 mg to about 1000 mg per administration, including about 250 mg to about 300 mg, about 300 mg to about 350 mg, about 350 mg to about 400 mg, about 400 mg to about 450 mg, about 450 mg to about 500 mg, about 500 mg to about 550 mg, about 550 mg to about 600 mg, about 600 mg to about 650 mg, about 650 mg to about 700 mg, about 700 mg to about 750 mg, about 750 mg to about 800 mg, about 800 mg to about 850 mg, about 850 mg to about 900 mg, about 900 mg to about 950 mg, about 950 mg to about 1000 mg, and overlapping ranges thereof.

When using a nutritional-based targeting moiety, concentrations that are effective to preferentially or specifically target bacteria can readily be determined. For example, in several embodiments, the concentration of about 1×10 M to about 1×10⁻³M of a sugar-based targeting moiety are used, including about 1×10 M to about 1×10⁻⁸M, about 1×10⁻⁸ M to about 1×10⁻⁷M, about 1×10⁻⁷ M to about 1×10⁻⁶M, about 1×10⁻⁶ M to about 1×10⁻⁵M, about 1×10⁻⁵ M to about 1×10⁻³M, about 1×10⁻⁴ M to about 1×10⁻³M, and overlapping ranges thereof.

Administration can be, for example, every other day, once per day, twice per day, three, four, five, six, or more times daily, or about every 72, 48, 36, 24, 18, 12, 8, 6, 4, 3 or 2 hours, or after every hemodialysis, to give some non-limiting examples, depending on the severity or type of infection, route of administration, hepatic and/or renal function of the patient, or other pharmacokinetic or clinical factors.

Routes of administration may also vary, depending on the embodiments. The complexed compositions are administered, in some embodiments, orally. In such embodiments, the composition can be formulated as any of capsules, chewable and dispersible tablets, syrups, suspensions, and the like. Delivery may also be subcutaneous, intramuscular, intravenous, intranasal, transdermal, topical, or intraperitoneal.

The duration of administration (e.g., the course of therapy) will vary from embodiment to embodiment, depending on the severity and/or type of infection. In several embodiments, an administration course will run from a one-time dose or 1 day, such as for prophylactic purposes, to just a few days to a week or more. In several embodiments, administration is for a frequency (as described above) for a duration of between about 5 and about 10 days, about 10 and about 14 days, about 14 and about 21 days, about 21 to about 31 days, about 1 month to about 3 months, about 3 to about 6 months, and times therebetween.

### Anti-Cancer Compounds

Second only to heart disease, cancer is a major cause of morbidity and mortality. Many current treatment regimens are expensive, lead to numerous adverse side effects and are, in essence, palliative treatments at best. The toll on society, medical providers, and families (both financial and emotional) is nearly incalculable. While chemotherapeutics and radiation therapy have made some cancers survivable, further improvements are needed.

As used herein, the term “cancer” and “cancerous” shall be given their ordinary meanings and shall also refer to or describe the physiological condition in mammals that is typically characterized by unregulated cell growth. Examples of cancer include, but are not limited to, carcinoma, lymphoma, sarcoma, blastoma and leukemia. More particular examples of such cancers include squamous cell carcinoma, lung cancer, pancreatic cancer, cervical cancer, bladder cancer, hepatoma, breast cancer, colon carcinoma, head and neck cancer, ovarian cancer and neuroblastoma. While the term “cancer” as used herein is not limited to any one specific form of the disease, it is believed that the methods of the invention can be effective for cancers which are found to be blood-related cancers and those cancers in which solid tumors form, including, but not limited to, multiple myeloma, mantle cell lymphoma and leukemias. Additionally, cancerous tissues that can be treated with the compositions disclosed herein include, but are not limited to acute lymphoblastic leukemia (ALL), acute myeloid leukemia (AML), adrenocortical carcinoma, Kaposi's sarcoma, lymphoma, gastrointestinal cancer, appendix cancer, central nervous system cancer, basal cell carcinoma, bile duct cancer, bladder cancer, bone cancer, brain tumors (including but not limited to astrocytomas, spinal cord tumors, brain stem glioma, craniopharyngioma, ependymoblastoma, ependymoma, medulloblastoma, medulloepithelioma, breast cancer, bronchial tumors, Burkitt's lymphoma, cervical cancer, colon cancer, chronic lymphocytic leukemia (CLL), chronic myelogenous leukemia (CML), chronic myeloproliferative disorders, ductal carcinoma, endometrial cancer, esophageal cancer, gastric cancer, Hodgkin's lymphoma, non-Hodgkin's lymphoma, hairy cell leukemia, renal cell cancer, leukemia, oral cancer, liver cancer, lung cancer, lymphoma, melanoma, ocular cancer, ovarian cancer, pancreatic cancer, prostate cancer, pituitary cancer, uterine cancer, and vaginal cancer.

### Cancer Targeting Moieties

Many cancer cell lines undergo rapid division, thus, they require a larger amount of nutrient delivery. In order to increase nutrient delivery, cancer cells can release growth factors that increase vascularization of tumor sites. This vascularization increases nutrient delivery to the cancer cells by increasing blood flow. In addition, cancer cells can also express larger amounts of nutrient receptors on their cell surface than would a non-cancer cell. For example, a cancer cell often overexpresses sugar receptors to increase the amount of sugar delivered into the cell. Because cancer cells overexpress nutrient receptors, cancer cells can be preferentially targeted over non-cancer cells by employing nutrient conjugates. Several embodiments of the present invention involve targeting cancer cells by exploiting increased nutrient receptors.

In several embodiments, a targeted anti-cancer conjugate is provided. In several embodiments, the anti-cancer conjugate comprises a nutrient-based targeting moiety. In several embodiments, the nutrient-based targeting moiety is functionally linked (e.g. associated or covalently linked) to an anti-cancer agent to provide a targeted anti-cancer conjugate. In several embodiments, the nutrient-based targeting moiety is a nutrient and/or an energy source for cancer cells. In several embodiments, the nutrient-based targeting moiety is an energy component which may include but is not limited to a number of nutrients including fructose, glucose, glutamine, glucosamine, among others, amino-acid-based moieties, and their functionalized derivatives. As used herein, the term “amino-acid-based moiety” shall be given its ordinary meaning and shall also refer to both standard and non-standard amino acids, including derivatives and analogs, halo and other heteroatoms. The term shall also refer to a side chain or group coming off the amino acid unit, typically alpha to the carboxyl group. Further still, in relevant instances, the term shall also include a single or series of bonded amino acid and/or amino alcohols with previously states groups substituted on said chain, including a combination of those groups. In several embodiments, the nutrient-based targeting moiety is selected from the group consisting of fructose, glucose, galactose, sucrose, maltose, lactose, alanine arginine, asparagine, aspartic acid, cysteine, glutamine, glutamic acid, glycine, histidine, isoleucine, leucine, lysine, methionine, phenylalanine, proline, serine, threonine, tryptophan, tyrosine, glucosamine, monosaccharides, dissaccharides, trisaccharides, oligosaccharides, polysaccharides, dipeptides, oligopeptides, polypeptides, proteins, and any combination thereof. In several embodiments, functionalized derivatives of any of these species may be employed. In several embodiments, vitamins and fatty acids may be used to target the cancer cells. In some embodiments, a functionalized derivative comprises an analog, a prodrug, or a derivitized version of the above species.

### Anti-Cancer Effector Moieties

Many cancers have a higher demand for nutrients, known as the Warburg effect, to aid proliferation and survival, which consequently leads to tumor growth that is more rapid than normal cells. For instance, cancer cells can uptake nutrients up to 200× or more greater than normal cells. This is a weakness of cancer that can be exploited therapeutically, as is done with several therapeutic compositions and methods disclosed herein.

Certain metal ions (e.g., silver ions) are known to interact with the mitochondria and/or DNA of cancer cells and thereby impart an anti-cancer effect. Inside the cell, silver binds to and disrupts the function of mitochondrial membranes, interfering with the energy (ATP) yielding reactions of the respiratory chain. Silver can also bind specifically to cellular enzymes and DNA, thus interfering with their functions. Thus, in several embodiments, ionic silver serves as the “warhead” which can be efficacious at inhibiting or killing cancer cells but which is non-toxic to normal, healthy cells. Several embodiments of the present invention advantageously capitalize on the mechanisms of action of silver ions (or other metal ions) in combination with increased uptake of the nutritional compound to which they are coupled, to provide unexpectedly efficacious anti-cancer effects.

In several embodiments, ionic silver is complexed to a sugar or amino acid moiety (as discussed above). In several embodiments, the preferential uptake of the anti-cancer targeting moiety by highly active cancer cells leads to a greater deposition of silver (or other metal ion or alternative effector moiety discussed below) in the cancer cells. As a result, the cancer cells are preferentially disrupted, leading to anti-cancer effects with limited (or non-existent) adverse effects on normal cells.

In several embodiments, other metals may be used in combination with the anti-cancer targeting moiety. For instance, in several embodiments, the anti-cancer targeting moiety is complexed to ions of mercury, copper, iron, lead, zinc, bismuth, gold, aluminum, or other metals. In several embodiments, Sc, Ti, V, Cr, Mn, Fe, Co, Ni, Cu, Zn, Ga, Rb, Sr, Y Zr, Nb, Mo, Tc, Ru, Rh, Pd, Cd, In, Sn, Cs, Ba, Ce, Pr, Nd, Pm, Sm, Eu, Gd, Tb, Dy, Ho, Er, Tm, Yb, Lu, Hf, Ta, W, Re, Os, Jr, Pt, Ag, Au, Hg, Tl, Pb, Bi, Po, Fr, Ra, Th, Pa, U Np, Pu, Am, Cm, Bk, Cf, Es, Fm, Md, No, Lr, Rf, Db, Sg, Bh, Hs, Mt, and/or combinations thereof may be used in combination with the anti-cancer targeting moiety. In several embodiments, combinations of one more type of anti-cancer targeting moiety and one or more of the above metals (in their ionic form) are used.

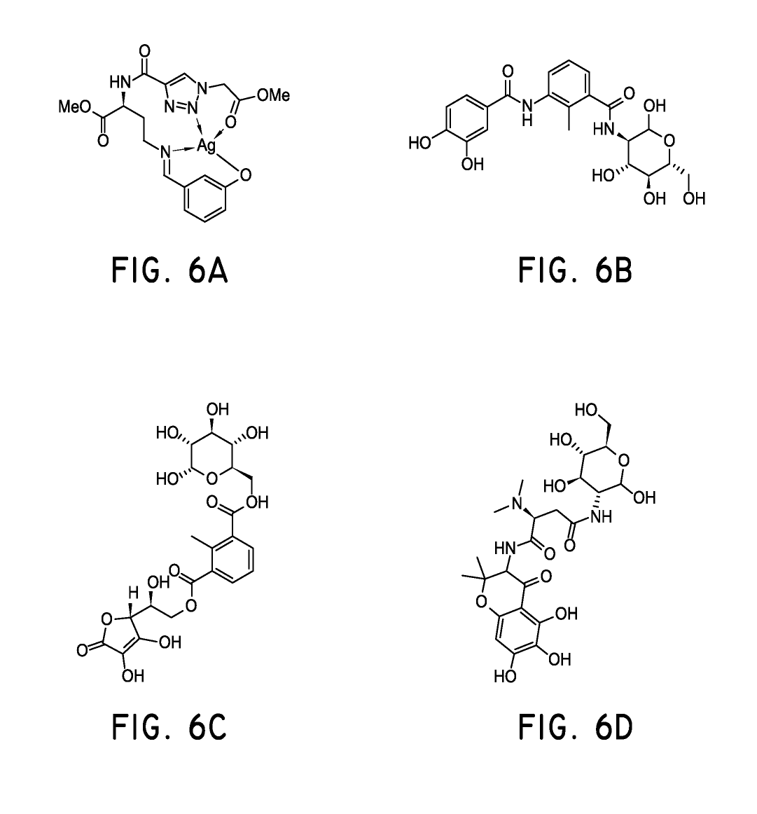

In several embodiments, rather than complexing a targeting moiety, such as a nutritional anti-cancer targeting moiety, to a metal ion, other anti-cancer effector moieties are used, either in place of, or in addition to a metal ion. For example, those molecules that can lead to generation of reactive oxygen species can be complexed to an anti-cancer targeting moiety and, upon administration to an individual with a tumor, generate localized reactive oxygen species, thus leading to inhibition and/or death of the tumor cells. For example, in several embodiments, compounds that promote the formation of superoxide ions are complexed to an anti-cancer targeting moiety. In several embodiments, generation of superoxide ions leads to one or more of DNA damage, mitochondrial dysfunction, increased apoptosis, each of which can occur in combination with any of the others, ultimately resulting in anti-tumor effects. In several embodiments, pro-oxidant compounds, such as for example vitamin C, zinc, vitamin E, and/or polyphenol antioxidants are used. It is surprising, in several embodiments, that molecules that are typically associated with antioxidant effects can be employed in a specifically targeted pro-oxidant capacity. However, for example, vitamin C (via the fenton reaction) can reduce metal ions and result in the generation of free radicals, leading to anti-cancer effects. Thus, in several embodiments, a composition comprising a mixture of targeting moieties complexed to metal ions and targeting moieties complexed to vitamin C result in synergistic anti-cancer effects. In additional embodiments, molecules that generate intracellular peroxides are complexed to anti-cancer targeting moieties. These molecules include those having one or more hydroxyl groups. In some embodiments, peroxide generating molecules that are known to be toxic to cells in other biological contexts are used to yield anti-cancer effects. For example, in several embodiments, one or more of pyocyanin (1-hydroxy-N-methylphenazine), dopamine, 6-hydroxydopamine, 6-aminodopamine, 6,7-dihydroxytryptamine, and dialuric acid are complexed to an anti-cancer targeting moiety. In several embodiments, prooxidant proteins are coupled to an anti-cancer targeting moiety. For example, in several embodiments, a member of the metallothionein family is complexed and results in production of hydroxyl radicals upon administration. In several embodiments, flavonoids including, but not limited to, flavones, isoflavones, and/or flavanones serve as prooxidants (particularly in the presence of copper ions) and are complexed to anti-cancer targeting moieties. In several embodiments, delivery of catechins, e.g., epigallocatechin or epicatechin generates hydrogen peroxide and/or hydroxyl radical that yield anti-cancer effects. In several embodiments, esters of fumaric acid (e.g., dimethyl fumarate or methylhydrogenfumarate) are complexed to an anti-cancer targeting moiety. In several embodiments, a peroxide generator, superoxide promoter, etc. is employed in conjunction with a nutritional based anti-cancer targeting moiety, instead of, or in addition to, a metal ion. Non-limiting embodiments of anti-cancer complexes are shown in FIG. 6A-6D

### Synthesis of Anti-Cancer Compounds

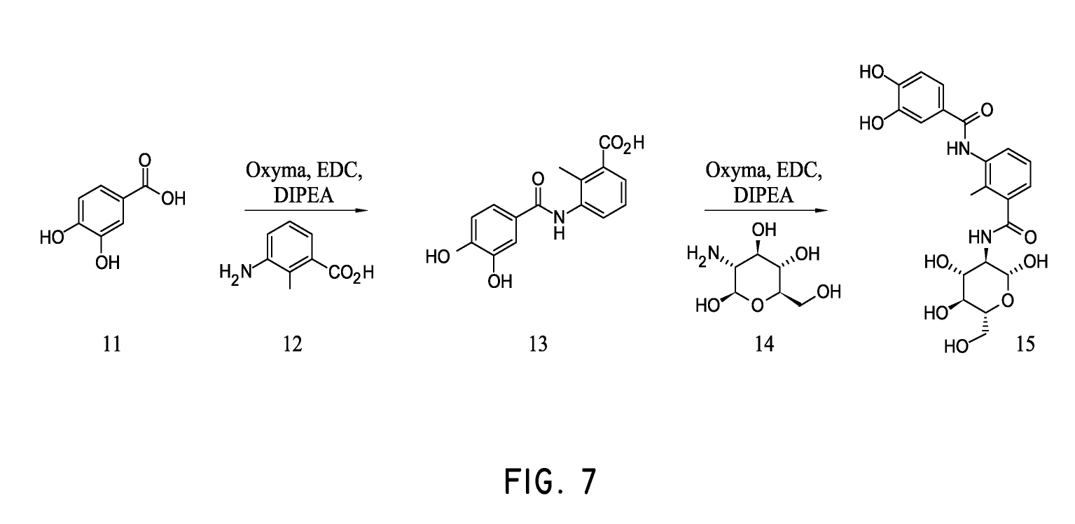

In one embodiment, shown as a non-limiting example in FIG. 7, 3-(3,4-dihydroxybenzamido)-2-methyl-N-((3R,4R,5S,6R)-2,4,5-trihydroxy-6-(hydroxymethyl)tetrahydro-2H-pyran-3-yl)benzamide (15) is synthesized by coupling 3,4-Dihydroxybenzoic acid (11) with 3-Amino-2-methylbenzoic acid (12) to obtain a peroxide generator intermediate (13). The peroxide generator intermediate (13) is then coupled with 2-amino-2-deoxy-beta-D-glucopyranose (14) providing target molecule 15.

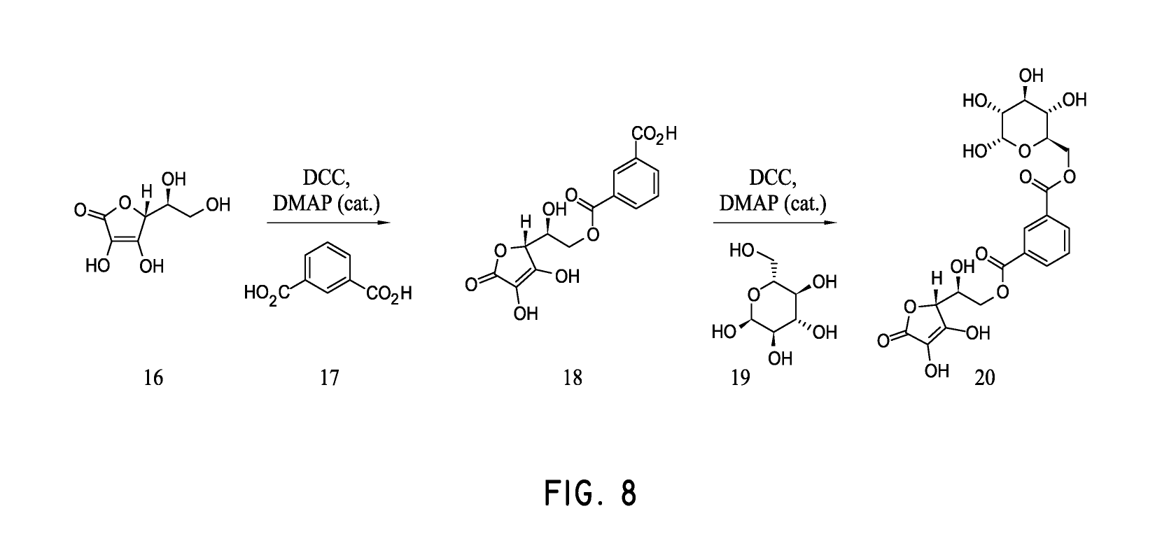

In another embodiment, shown as a non-limiting example in FIG. 8, (S)-2-((R)-3,4-dihydroxy-5-oxo-2,5-dihydrofuran-2-yl)-2-hydroxyethyl(((2R,3S,4S,5R,6S)-3,4,5,6-tetrahydroxytetrahydro-2H-pyran-2-yl)methyl) isophthalate (20) is synthesized by coupling vitamin C (16) with 3-Formylbenzoic acid (17) to obtain peroxide generator intermediate (18). The peroxide generator intermediate (18) is then coupled with D-(+)-Glucose (19) providing target molecule 20.

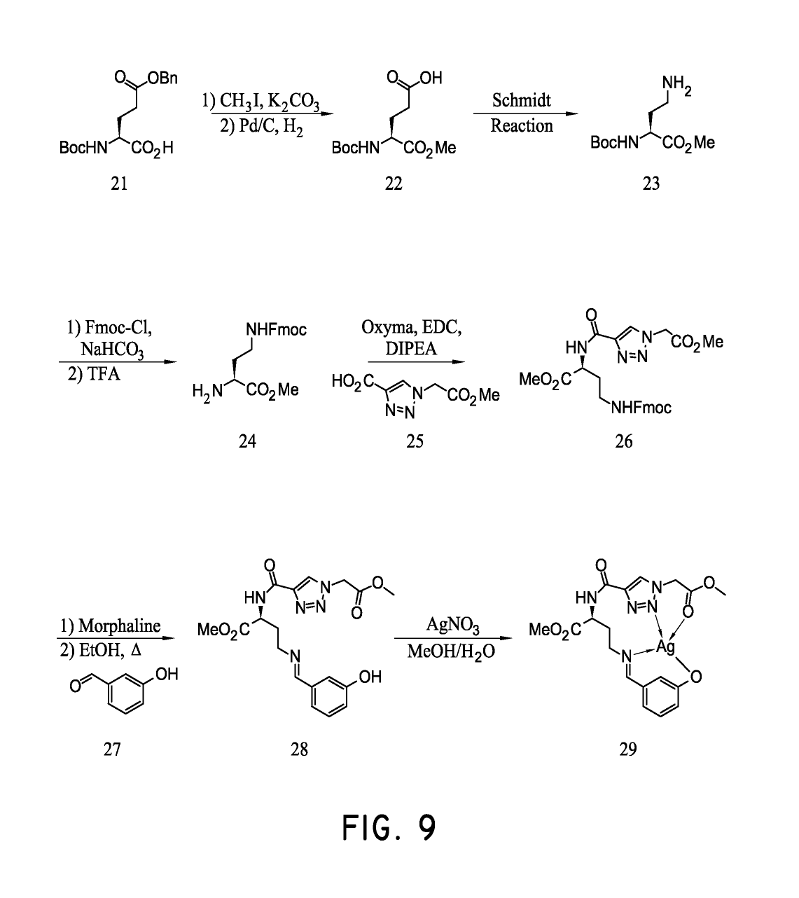

In yet another embodiment, shown as a non-limiting example in FIG. 9, (S,E)-methyl 4-((3-hydroxybenzylidene)amino)-2-(1-(2-methoxy-2-oxoethyl)-1H-1,2,3-triazole-4-carboxamido)butanoate silver complex (29) is synthesized by methylating (S)-5-(benzyloxy)-2-((tert-butoxycarbonyl)amino)-5-oxopentanoic acid (21) followed by deprotecting the benzyl group forming (S)-4-((tert-butoxycarbonyl)amino)-5-methoxy-5-oxopentanoic acid (22). Acid 22 is submitted to the Schmidt Reaction conditions (or similar reaction) to obtain (S)-methyl 4-amino-2-((tert-butoxycarbonyl)amino)butanoate (23). After protection of the free amine with orthogonal protecting group, 9-Fluorenylmethyl chloroformate, the boc protected amine is deprotected providing (S)-methyl 4-((((9H-fluoren-9-yl)methoxy)carbonyl)amino)-2-aminobutanoate (24). Coupling amine 24 with 1-(2-methoxy-2-oxoethyl)-1H-1,2,3-triazole-4-carboxylic acid (25) provides (S)-methyl 4-((((9H-fluoren-9-yl)methoxy)carbonyl)amino)-2-(1-(2-methoxy-2-oxoethyl)-1H-1,2,3-triazole-4-carboxamido)butanoate (26). After deprotecting the Fmoc protecting group on triazole 26, the amine is treated with 3-Hydroxybenzaldehyde (27) producing (S,E)-methyl 4-((3-hydroxybenzylidene)amino)-2-(1-(2-methoxy-2-oxoethyl)-1H-1,2,3-triazole-4-carboxamido)butanoate (28) which is finally treated with silver nitrate to obtain the target molecule 29.

### Treatment of Cancers

The treatment of various cancers using the compositions disclosed herein can be achieved in a variety of ways, depending on the embodiment. When using a nutritional-based targeting moiety, concentrations that are effective to preferentially or specifically target bacteria can readily be determined. For example, in several embodiments, the concentration of about 1×10⁻⁹ M to about 1×10⁻³M of a sugar-based targeting moiety are used, including about 1×10⁻⁹ M to about 1×10⁻⁸ M, about 1×10⁻⁸ M to about 1×10⁻⁷M, about 1×10⁻⁷ M to about 1×10⁻⁶M, about 1×10⁻⁶ M to about 1×10⁻⁵M, about 1×10⁻⁵ M to about 1×10⁻³M, about 1×10⁻⁴ M to about 1×10⁻³M, and overlapping ranges thereof.

Administration can be once per day, twice per day, three, four, five, six, or more times daily, depending on the severity of the cancer and other relevant clinical factors. Drug-drug interactions, and possible adverse effects, are also taken into account in several embodiments.

Routes of administration may also vary, depending on the embodiments. The complexed compositions are administered, in some embodiments, orally. In such embodiments, the composition can be formulated as any of capsules, chewable and dispersible tablets, syrups, suspensions, and the like. Delivery may also be subcutaneous, intramuscular, intravenous, or intraperitoneal.

The duration of administration (e.g., the course of therapy) will vary from embodiment to embodiment, depending on the severity and/or type of cancer, its location, and its aggressiveness. In several embodiments, an administration course will run from several weeks to several months. For example, in several embodiments the compositions are administered at a frequency (as described above) for a duration of between about 3 weeks and about 6 weeks, about 6 and about 10 weeks, about 2 and about 4 weeks, about 1 and about 5 weeks and overlapping ranges thereof. In several embodiments, the administration is given in “courses”, e.g., administration for a period of weeks, followed by a recovery period without administration, followed by a further administration period.

In several embodiments of the invention, the compounds of the present invention can be administered as the sole anti-cancer agent. However, in several embodiments, the compositions are used in combination with one or more adjunctive therapies (e.g., chemo, hormonal therapy, surgery, radiation, etc.).

The phrase “co-therapy” (or “combination-therapy”), in defining use of a compound disclosed herein with at least one other pharmaceutical agent, is intended to embrace administration of each agent in a sequential manner in a regimen that will provide beneficial effects of the drug combination, and is intended as well to embrace co-administration of these agents in a substantially simultaneous manner, such as in a single dose having a fixed ratio of these active agents or in multiple, separate doses for each agent.

Specifically, the administration of the compounds disclosed herein can be in conjunction with additional therapies known to those skilled in the art in the prevention or treatment of neoplastic disease, such as with radiation therapy or with cytostatic or cytotoxic agents.

Standard treatment of primary tumors can include surgical excision followed by either radiation or intravenously (IV) administered chemotherapy. The typical chemotherapy regime can include either DNA alkylating agents, DNA intercalating agents, CDK inhibitors, or microtubule poisons. The chemotherapy doses used are just below the maximal tolerated dose and therefore dose limiting toxicities typically include, nausea, vomiting, diarrhea, hair loss, neutropenia and the like.

A large number of antineoplastic agents are available in commercial use, in clinical evaluation and in pre-clinical development, which can be selected for treatment of neoplastic disease by combination drug chemotherapy. Such antineoplastic agents fall into several major categories, namely, antibiotic-type agents, alkylating agents, antimetabolite agents, hormonal agents, immunological agents, interferon-type agents and a category of miscellaneous agents.

A first family of antineoplastic agents which can be used in combination with embodiments of the invention disclosed herein comprises antimetabolite-type/thymidilate synthase inhibitor antineoplastic agents. Suitable antimetabolite antineoplastic agents can be selected from, but are not limited to, the group consisting of 5-FU-fibrinogen, acanthifolic acid, aminothiadiazole, brequinar sodium, cammofur, Ciba-Geigy CGP-30694, cyclopentyl cytosine, cytarabine phosphate stearate, cytarabine conjugates, Lilly DATHF, Merrel Dow DDFC, dezaguanine, dideoxycytidine, dideoxyguanosine, didox, Yoshitomi DMDC, doxifluridine, Wellcome EHNA, Merck & Co. EX-015, fazarabine, floxuridine, fludarabine phosphate, 5-fluorouracil, N-(2′-furanidyl)-5-fluorouracil, Daiichi Seiyaku FO-152, isopropyl pyrrolizine, Lilly LY-188011, Lilly LY-264618, methobenzaprim, methotrexate, Wellcome MZPES, norspermidine, NCI NSC-127716, NCI NSC-264880, NCI NSC-39661, NCI NSC-612567, Warner-Lambert PALA, pentostatin, piritrexim, plicamycin, Asahi Chemical PL-AC, Takeda TAC-788, thioguanine, tiazofurin, Erbamont TIF, trimetrexate, tyrosine kinase inhibitors, Taiho UFT and uricytin.

A second family of antineoplastic agents which can be used in combination with embodiments of the invention disclosed herein comprises alkylating-type antineoplastic agents. Suitable alkylating-type antineoplastic agents can be selected from, but not limited to, the group consisting of Shionogi 254-S, aldo-phosphamide analogues, altretamine, anaxirone, Boehringer Mannheim BBR-2207, bestrabucil, budotitane, Wakunaga CA-102, carboplatin, carmustine, Chinoin-139, Chinoin-153, chlorambucil, cisplatin, cyclophosphamide, American Cyanamid CL-286558, Sanofi CY-233, cyplatate, Degussa D-19-384, Sumitomo DACHP(Myr)2, diphenylspiromustine, diplatinum cytostatic, Erba distamycin derivatives, Chugai DWA-2114R, ITI E09, elmustine, Erbamont FCE-24517, estramustine phosphate sodium, fotemustine, Unfitted G-6-M, Chinoin GYKI-17230, hepsul-fam, Ifosfamide, iproplatin, lomustine, mafosfamide, mitolactol, Nippon Kayaku NK-121, NCI NSC-264395, NCI NSC-342215, oxaliplatin, Upjohn PCNU, prednimustine, Proter PTT-119, ranimustine, semustine, SmithKline SK&F-101772, Yakult Honsha SN-22, spiromus-tine, Tanabe Seiyaku TA-077, tauromustine, temozolomide, teroxirone, tetraplatin and trimelamol.

A third family of antineoplastic agents which can be used in combination with embodiments of the invention disclosed herein comprises antibiotic-type antineoplastic agents. Suitable antibiotic-type antineoplastic agents can be selected from, but are not limited to, the group consisting of Taiho 4181-A, aclarubicin, actinomycin D, actinoplanone, Erbamont ADR-456, aeroplysinin derivative, Ajinomoto AN-201-1, Ajinomoto AN-3, Nippon Soda anisomycins, anthracycline, azino-mycin-A, bisucaberin, Bristol-Myers BL-6859, Bristol-Myers BMY-25067, Bristol-Myers BMY-25551, Bristol-Myers BMY-26605, Bristol-Myers BMY-27557, Bristol-Myers BMY-28438, bleomycin sulfate, bryostatin-1, Taiho C-1027, calichemycin, chromoximycin, dactinomycin, daunorubicin, Kyowa Hakko DC-102, Kyowa Hakko DC-79, Kyowa Hakko DC-88A, Kyowa Hakko DC89-A1, Kyowa Hakko DC92-B, ditrisarubicin B, Shionogi DOB-41, doxorubicin, doxorubicin-fibrinogen, elsamicin-A, epirubicin, crbstatin, esorubicin, esperamicin-A1, esperamicin-A1b, Erbamont FCE-21954, Fujisawa FK-973, fostriecin, Fujisawa FR-900482, glidobactin, gregatin-A, grincamycin, herbimycin, idarubicin, illudins, kazusamycin, kesarirhodins, Kyowa Hakko KM-5539, Kirin Brewery KRN-8602, Kyowa Hakko KT-5432, Kyowa Hakko KT-5594, Kyowa Hakko KT-6149, American Cyanamid LL-D49194, Meiji Seika ME 2303, menogaril, mitomycin, mitoxantrone, SmithKline M-TAG, neoenactin, Nippon Kayaku NK-313, Nippon Kayaku NKT-01, SRI International NSC-357704, oxalysine, oxaunomycin, peplomycin, pilatin, pirarubicin, porothramycin, pyrindanycin A, Tobishi RA-I, rapamycin, rhizoxin, rodorubicin, sibanomicin, siwenmycin, Sumitomo SM-5887, Snow Brand SN-706, Snow Brand SN-07, sorangicin-A, sparsomycin, SS Pharmaceutical SS-21020, SS Pharmaceutical SS-7313B, SS Pharmaceutical SS-9816B, steffimycin B, Taiho 4181-2, talisomycin, Takeda TAN-868A, terpentecin, thrazine, tricrozarin A, Upjohn U-73975, Kyowa Hakko UCN-10028A, Fujisawa WF-3405, Yoshitomi Y-25024, zorubicin, peptide boronates (e.g. bortezomib), α′β′-epoxyketones (e.g. epoxomoxin), β-lactones (e.g. salinosporamide A, salinosporamide B, fluorosalinosporamide, lactacystin), cinnabaramide A, cinnabaramide B, cinnabaramide C, belactosines (e.g. homobelactosin C), fellutamide B, TMC-95A, PS-519, omuralide, and antiprotealide ‘Salinosporamide-Omularide Hybrid.’

A fourth family of antineoplastic agents which can be used in combination with embodiments of the invention disclosed herein comprises a miscellaneous family of antineoplastic agents, including, but not limited to, tubulin interacting agents, topoisomerase II inhibitors, topoisomerase I inhibitors and hormonal agents, selected from but not limited to the group consisting of a-carotene, a-difluoromethyl-arginine, acitretin, Biotec AD-5, Kyorin AHC-52, alstonine, amonafide, amphethinile, amsacrine, Angiostat, ankinomycin, anti-neoplaston A10, antineoplaston A2, antineoplaston A3, antineoplaston A5, antineoplaston AS2-1, Henkel APD, aphidicolin glycinate, asparaginase, Avarol, baccharin, batracylin, benfluoron, benzotript, Ipsen-Beaufour BIM-23015, bisantrene, Bristol-Myers BMY-40481, Vestar boron-10, bromofosfamide, Wellcome BW-502, Wellcome BW-773, caracemide, carmethizole hydrochloride, Ajinomoto CDAF, chlorsulfaquinoxalone, Chemes C1H-2053, Chemex CHX-100, Warner-Lambert CI-921, Warner-Lambert CI-937, Warner-Lambert CI-941, Warner-Lambert CI-958, clanfenur, claviridenone, ICN compound 1259, ICN compound 4711, Contracan, Yakult Honsha CPT-11, crisnatol, curaderm, cytochalasin B, cytarabine; cytocytin, Merz D-609, DABIS maleate, dacarbazine, datelliptinium, didemnin-B, dihaematoporphyrin ether, dihydrolenperone, dinaline, distamycin, Toyo Pharmar DM-341, Toyo Pharmar DM-75, Daiichi Seiyaku DN-9693, docetaxel elliprabin, elliptinium acetate, Tsumura EPMTC, the epothilones, ergotamine, etoposide, etretinate, fenretinide, Fujisawa FR-57704, gallium nitrate, genkwadaphnin, Chugai GLA-43, Glaxo GR-63178, grifolan NMF-5N, hexadecylphosphocholine, Green Cross HO-221, homoharringtonine, hydroxyurea, BTG ICRF-187, ilmofosine, isoglutamine, isotretinoin, Otsuka Ramot K-477, Otsuak K-76COONa, Kureha Chemical K-AM, MECT Corp KI-8110, American Cyanamid L-623, leukoregulin, lonidamine, Lundbeck LU-23-112, Lilly LY-186641, NCI (US) MAP, marycin, Merrel Dow MDL-27048, Medco MEDR-340, merbarone, merocyanlne derivatives, methylanilinoacridine, Molecular Genetics MGI-136, minactivin, mitonafide, mitoquidone mopidamol, motretinide, Zenyaku Kogyo MST-16, N-(retinoyl)amino acids, Nisshin Flour Milling N-021, N-acylated-dehydroalanines, nafazatrom, Taisho NCU-190, nocodazole derivative, Normosang, NCI NSC-145813, NCI NSC-361456, NCI NSC-604782, NCI NSC-95580, ocreotide, Ono ONO-112, oquizanocine, Akzo Org-10172, paclitaxel, pancratistatin, pazelliptine, Warner-Lambert PD-111707, Warner-Lambert PD-115934, Warner-Lambert PD-131141, Pierre Fabre PE-1001, ICRT peptide D, piroxantrone, polyhaematoporphyrin, polypreic acid, Efamol porphyrin, probimane, procarbazine, proglumide, Invitron protease nexin I, Tobishi RA-700, razoxane, Sapporo Breweries RBS, restrictin-P, retelliptine, retinoic acid, Rhone-Poulenc RP-49532, Rhone-Poulenc RP-56976, SmithKline SK&F-104864, Sumitomo SM-108, Kuraray SMANCS, SeaPharm SP-10094, spatol, spirocyclopropane derivatives, spirogermanium, Unimed, SS Pharmaceutical SS-554, strypoldinone, Stypoldione, Suntory SUN 0237, Suntory SUN 2071, superoxide dismutase, Toyama T-506, Toyama T-680, taxol, Teijin TEI-0303, teniposide, thaliblastine, Eastman Kodak TJB-29, tocotrienol, topotecan, Topostin, Teijin TT-82, Kyowa Hakko UCN-01, Kyowa Hakko UCN-1028, ukrain, Eastman Kodak USB-006, vinblastine sulfate, vincristine, vindesine, vinestramide, vinorelbine, vintriptol, vinzolidine, with anolides and Yamanouchi YM-534.

In some embodiments, the compounds disclosed herein can be used in co-therapies with other anti-neoplastic agents, such as acemannan, aclarubicin, aldesleukin, alemtuzumab, alitretinoin, altretamine, amifostine, aminolevulinic acid, amrubicin, amsacrine, anagrelide, anastrozole, ANCER, ancestim, ARGLABIN, arsenic trioxide, RAM 002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alpha, daunorubicin, doxorubicin, tretinoin, edelfosine, edrecolomab, eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alpha, interferon alpha, natural, interferon alpha-2, interferon alpha-2a, interferon alpha-2b, interferon alpha-N1, interferon alpha-n3, interferon alfacon-1, interferon alpha, natural, interferon beta, interferon beta-1a, interferon beta-1b, interferon gamma, natural interferon gamma-1a, interferon gamma-1b, interleukin-1 beta, iobenguane, irinotecan, irsogladine, lanreotide, LC 9018 (Yakult), leflunomide, lenograstim, lentinan sulfate, letrozole, leukocyte alpha interferon, leuprorelin, levamisole+fluorouracil, liarozole, lobaplatin, lonidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alpha-2b, pentosan polysulfate sodium, pentostatin, picibanil, pirarubicin, rabbit antithymocyte polyclonal antibody, polyethylene glycol interferon alpha-2a, porfimer sodium, raloxifene, raltitrexed, rasburicase, rhenium Re 186 etidronate, RII retinamide, rituximab, romurtide, samarium (153 Sm) lexidronam, sargramostim, sizofuran, sobuzoxane, sonermin, strontium-89 chloride, suramin, tasonermin, tazarotene, tegafur, temoporfin, temozolomide, teniposide, tetrachlorodecaoxide, thalidomide, thymalfasin, thyrotropin alpha, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, VIRULIZIN, zinostatin stimalamer, or zoledronic acid; abarelix; AE 941 (Aeterna), ambamustine, antisense oligonucleotide, bcl-2 (Genta), APC 8015 (Dendreon), cetuximab, decitabine, dexaminoglutethimide, diaziquone, EL 532 (Elan), EM 800 (Endorecherche), eniluracil, etanidazole, fenretinide, filgrastim SD01 (Amgen), fulvestrant, galocitabine, gastrin 17-immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb) (Trilex), LYM-1-iodine 131 MAb (Techniclone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), melanoma vaccine (New York University), melanoma vaccine (Sloan Kettering Institute), melanoma oncolysate vaccine (New York Medical College), viral melanoma cell lysates vaccine (Royal Newcastle Hospital), valspodar, or proteasome inhibitors, including, but not limited to, peptide aldehydes (such as, for example, calpain inhibitor I/II, MG132), peptide boronates (such as, for example, Velcade/bortezomib, CEP-18770), β-lactones (such as, for example, lactacystin, Salinosporamide A/B, NPI-0052), peptide vinyl sulfones (such as, for example, NLVS, YLVS, ZLVS), and peptide epoxylketones (such as, for example, epoxomycin, TMC, carfilzomib).

In some embodiments, the compounds disclosed herein can be used in co-therapies with other agents, such as other kinase inhibitors including p38 inhibitors and CDK inhibitors, TNF inhibitors, metallomatrix proteases inhibitors (MMP), COX-2 inhibitors including celecoxib, rofecoxib, parecoxib, valdecoxib, and etoricoxib, NSAID's, SOD mimics or αvβ3 inhibitors, and anti-inflammatories.

In some embodiments, one, two, or more anti-microbial and/or anti-cancer conjugates can be complexed, coated, or otherwise operatively associated with a temporarily or permanently implanted medical device to prevent or treat infection, such as a catheter (including centrally or peripherally inserted intravenous, hemodialysis, peritoneal dialysis, or other catheters and shunts, stents, pacemakers and their leads, automatic internal converter defibrillators, prosthetic grafts, meshes, sutures, implantable beads, and other implants.

### EXAMPLES

### Example 1

### Synthesis of an Antibiotic-Silver Conjugate

This synthesis scheme is shown in FIG. 4. (6R,7R)-3-(acetoxymethyl)-7-((Z)-2-(2-(((Z)-(2-hydroxycyclohexa-2,4-dien-1-ylidene)methyl)amino)thiazol-4-yl)-2-(methoxyimino)acetamido)-8-oxo-5-thia-1-azabicyclo[4.2.0]oct-2-ene-2-carboxylic acid silver complex (10), is synthesized by nitrosation of acetoacetic ester (1) with nitrous acid which produces isonitrosoacetoacetic ester (2). O-Methylation of the hydroxyl group of the obtained product with dimethylsulfate in the presence of potassium carbonate provides ethyl 2-(methoxyimino)acetoacetate (3). Brominating the resulting product with bromine in methylene chloride in the presence of p-toluenesulfonic acid provides 4-bromo-2-methoxyiminoacetoacetic ester (4). Reacting 4 with thiourea according to the classic scheme of preparing of thiazoles from α-bromocarbonyl compounds and thioamides provides the ethyl ester of 2-(2-amino-4-thiazolyl)-2-methoxyiminoacetic acid (5). Reacting 5 with triphenylchloromethane in the presence of triethylamine results in a trityl protection of the amino group, forming the ethyl ester of 2-(2-tritylamino-4-thiazolyl)-2-methoxyminoacetic acid (6), which is hydrolyzed to the acid (7) using sodium hydroxide. The resulting acid 7 is used for acylating of 7-aminocephalosporanide acid in the presence of dicyclohexylcarbodiimide (DCC), giving tritylated cefotaxime, α-O-methyloxime acetate 7-[2-(2-tritylamino)-4-thiazolyl-glycoxylamido]-3-(hydroxymethyl)-8-oxo-5-thia-1-azabicyclo[4.2.0]oct-2-en-2-carboxylic acid (8). Finally, removing the trityl protection from the synthesized product (8) using dilute formic acid gives cefotaxime. [Synthesis of Essential Drugs, ISBN: 978-0-444-52166-8]. Cefotaxime is coupled with 2-hydroxybenzaldehyde to provide (6R,7R)-3-(acetoxymethyl)-7-((Z)-2-(2-(((Z)-(2-hydroxycyclohexa-2,4-dien-1-ylidene)methyl)amino)thiazol-4-yl)-2-(methoxyimino)acetamido)-8-oxo-5-thia-1-azabicyclo[4.2.0]oct-2-ene-2-carboxylic acid (9). The resulting acid 9 is complex with silver nitrate to provide (6R,7R)-3-(acetoxymethyl)-7-((Z)-2-(2-(((Z)-(2-hydroxycyclohexa-2,4-dien-1-ylidene)methyl)amino)thiazol-4-yl)-2-(methoxyimino)acetamido)-8-oxo-5-thia-1-azabicyclo[4.2.0]oct-2-ene-2-carboxylic acid silver complex (10) or cefotaxime derivative silver complex.

### Example 2

### Antimicrobial Efficacy of Antibiotic-Silver Conjugates

Cefazolin sodium salt can be subjected to silver nitrate to provide a cefazolin silver complex (e.g., as shown in FIG. 3A). This complex can be easily compared to the cefazolin sodium salt by testing the minimum inhibitory concentration (MIC). Initial tests were performed against a drug resistant gram negative bacterium; carbapenem-resistant enterobacteriaceae (CRE) and a drug resistant gram positive bacterium; methicillin-resistant staphylococcus aureus (MRSA). The associated MIC values for cefazolin sodium salt were >256 μg/ml for CRE and 256 μg/ml for MRSA, but when performed against the cefazolin silver complex the MIC values were 4 μg/ml for CRE and 4 μg/ml for MRSA. This unexpectedly large improvement by the ionic silver complex demonstrates how, in several embodiments, the beta-lactam moiety (or other antimicrobial targeting moiety) serves not only to stabilize the ionic silver (or other metal) but also provides an active mode delivery system to the bacterium. By transforming the cefazolin sodium salt into a silver complex, it changes the antibiotic that CRE and MRSA are resistant to, to a compound that CRE and MRSA are susceptible to. Thus, in several embodiments, the compositions provided for herein lead to a heretofore unseen efficacy against drug-resistant microorganisms.

### Example 3

### Synthesis of an Anti-Cancer Conjugate

An anti-cancer conjugate may be formed, for example according to the scheme shown in FIG. 7. According to that scheme, 3-(3,4-dihydroxybenzamido)-2-methyl-N-((3R,4R,5S,6R)-2,4,5-trihydroxy-6-(hydroxymethyl)tetrahydro-2H-p yran-3-yl)benz amide (15) is synthesized by coupling 3,4-Dihydroxybenzoic acid (11) with 3-Amino-2-methylbenzoic acid (12) to obtain a peroxide generator intermediate (13). The peroxide generator intermediate (13) is then coupled with 2-amino-2-deoxy-beta-D-glucopyranose (14) providing target molecule 15.

### Example 4

### Synthesis of an Anti-Cancer Conjugate

An anti-cancer conjugate may be formed, for example according to the scheme shown in FIG. 8. According to that scheme, (S)-2-((R)-3,4-dihydroxy-5-oxo-2,5-dihydrofuran-2-yl)-2-hydroxyethyl(((2R,3S,4S,5R,6S)-3,4,5,6-tetrahydroxytetrahydro-2H-pyran-2-yl)methyl) isophthalate (20) is synthesized by coupling vitamin C (16) with 3-Formylbenzoic acid (17) to obtain peroxide generator intermediate (18). The peroxide generator intermediate (18) is then coupled with D-(+)-Glucose (19) providing target molecule 20.

### Example 5

### Synthesis of an Anti-Cancer Conjugate

An anti-cancer conjugate may be formed, for example according to the scheme shown in FIG. 9. According to that scheme, (S,E)-methyl 4-((3-hydroxybenzylidene)amino)-2-(1-(2-methoxy-2-oxoethyl)-1H-1,2,3-triazole-4-carboxamido)butanoate silver complex (29) is synthesized by methylating (S)-5-(benzyloxy)-2-((tert-butoxycarbonyl)amino)-5-oxopentanoic acid (21) followed by deprotecting the benzyl group forming (S)-4-((tert-butoxycarbonyl)amino)-5-methoxy-5-oxopentanoic acid (22). Acid 22 is submitted to the Schmidt Reaction conditions (or similar reaction) to obtain (S)-methyl 4-amino-2-((tert-butoxycarbonyl)amino)butanoate (23). After protection of the free amine with orthogonal protecting group, 9-Fluorenylmethyl chloroformate, the boc protected amine is deprotected providing (S)-methyl 4-((((9H-fluoren-9-yl)methoxy)carbonyl)amino)-2-aminobutanoate (24). Coupling amine 24 with 1-(2-methoxy-2-oxoethyl)-1H-1,2,3-triazole-4-carboxylic acid (25) provides (S)-methyl 4-((((9H-fluoren-9-yl)methoxy)carbonyl)amino)-2-(1-(2-methoxy-2-oxoethyl)-1H-1,2,3-triazole-4-carboxamido)butanoate (26). After deprotecting the Fmoc protecting group on triazole 26, the amine is treated with 3-Hydroxybenzaldehyde (27) producing (S,E)-methyl 4-((3-hydroxybenzylidene)amino)-2-(1-(2-methoxy-2-oxoethyl)-1H-1,2,3-triazole-4-carboxamido)butanoate (28) which is finally treated with silver nitrate to obtain the target molecule 29.

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “administering a silver-complexed antibiotic” include “instructing the administration of a silver-complexed antibiotic.” The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 3 mm” includes “3 mm.”

1. An anti-bacterial conjugate, comprising:
a targeting antibiotic; and
an anti-bacterial agent, wherein the anti-bacterial agent has generalized anti-bacterial activity, wherein the anti-bacterial agent is an agent to which bacteria do not develop resistance.

2.-35. (canceled)
